(19) United States
(12) Patent Application Publication
(10) Pub. No.: US 2016/0296435 A1
(43) Pub. Date: Oct. 13, 2016

(54) AGENT AND METHOD FOR THE TEMPORARY SHAPING OF KERATIN FIBERS

(71) Applicant: Henkel AG & Co. KGaA, Duesseldorf (DE)

(72) Inventors: Jisook Baek, Shanghai (CN); Anna Puls, Winsen (DE)

(73) Assignee: Henkel AG & Co. KGaA, Duesseldorf (DE)

(21) Appl. No.: 15/186,290

(22) Filed: Jun. 17, 2016

Related U.S. Application Data
(63) Continuation of application No. PCT/DE2014/200537, filed on Oct. 8, 2014.

(30) Foreign Application Priority Data
Dec. 23, 2013 (DE) 10 2013 227 116.6

(51) Int. Cl. A61K 8/25; A61K 8/19; A61K 8/81; A61K 8/88; A61Q 5/06; A61K 8/02; A61K 8/34

(52) U.S. Cl. CPC A61K 8/25; A61K 8/022; A61K 8/027; A61K 8/19; A61K 8/345; A61K 8/88; A61Q 5/06; A61Q 5/065; A61K 8/8158; A61K 2800/436; A61K 2800/54; A61K 2800/591; A61K 2800/26

(57) ABSTRACT

The invention relates to powdered cosmetic compositions, containing a) 10 to 30% by weight of a hydrophobized metal oxide powder, b) 40 to 89% by weight of a polar solvent, and c) 0.001 to 8.0% by weight of a polymer fiber material. Said compositions are particularly suitable for reshaping and scenting keratin fibers and provide shine, texture, and silkiness.

### FIELD OF THE INVENTION

The present invention generally relates to the technical field of the temporary reshaping of keratin-containing fibers, in particular human hair.

### BACKGROUND OF THE INVENTION

Styling agents for reshaping keratin-containing fibers have been known for some time and are used in various forms for volumizing, refreshing, and retaining of hairstyles, which for many hair types can only be achieved using setting active substances. Both hair treatment agents used to shape hair permanently and those that shape it temporarily play an important role in this respect. Temporary shaping, which is intended to provide a good hold without impairing the healthy appearance of the hair, such as, for example, the shine thereof, can be achieved, for example, by hairsprays, hair waxes, hair gels, blow-dry waves, etc.

Suitable agents for temporary shaping usually include synthetic polymers as a shaping component. Preparations including a polymer can be applied to the hair by means of propellant gases or by a pump mechanism. Hair gels and hair waxes, on the other hand, are generally applied onto the hair not directly but are distributed in the hair by means of a comb or the hands.

Known forms of temporary styling agents often cannot be dispensed with satisfactory accuracy. Thus, for instance, hair gels, hair creams, and hair waxes are difficult to distribute, once they have been applied to the hair.

Hairsprays can be applied to the hair more uniformly. But because it is not possible for the user to visually determine the total amount of applied styling agent, there is the risk that more styling agent than would actually be necessary is applied to the hair.

Powdered cosmetics are known and have already been used for some time, for instance, in the field of skin treatment. Typical examples, for instance, are makeup powder or eyeshadow. To achieve the powdered consistency, it is necessary to use a powdered carrier material. For instance, a metal oxide such as, e.g., silicon dioxide can be used as a suitable carrier material. Of particular interest is hydrophobized metal oxide or silicon dioxide. This can be obtained, for example, from pyrogenic silicon dioxide, which is commercially obtainable in various specifications. Untreated pyrogenic silicon dioxide carries silanol groups and siloxane groups on the surface. As a result, it has a high affinity for water; i.e., it is hydrophilic. By reaction with suitable organic silicon compounds, alkylsilyl groups can be chemically bound to the surface of the pyrogenic silicon dioxide. Modified silicon dioxide powders are formed that can no longer be wetted by water, i.e., that have hydrophobic properties. This hydrophobized silicon dioxide is suitable for preparing so-called dry water, in which the water droplets are prevented from coalescing again. The resulting powdered solids can have a water content of up to 95%. The enclosed water is released again under mechanical stress, for example, upon being rubbed onto the skin.

Cosmetic or pharmaceutical liquefiable powder compositions are described, for example, in European patent EP 1235554 B1.

The international application WO 03/037287 A1 discloses the use of granules based on pyrogenic silicon dioxide in cosmetic compositions. The special granules can be silanized, i.e., hydrophobized, and are suitable for preparing cosmetic compositions of any consistency, for example, liquids, foams, sprays, or powders.

The international application WO 2007/051511 A1 describes the use of a powdered composition, including 50 to 95% by weight of an aqueous solvent, hydrophobized silicon dioxide powder, and a film-forming and/or setting polymer, present at least in the aqueous solvent, for the temporary reshaping of keratinic fibers.

The subject of the German patent application DE 102008057261 A1 is powdered compositions which are used for the temporary reforming of hair for a very strong hold of the set hairstyle.

The powdered hair cosmetics of the state of the art meanwhile do in fact provide a hold acceptable for hair reforming and are notable for good dispensability. Nevertheless, the result achieved with these agents in regard to the parameters of natural shine and elasticity and hold is also in need of improvement as is their applicability, for example, by application to hands and hair. It is desirable simultaneously to achieve with the use of powdered products not only a high hairstyle hold but in addition an increased hairstyle volume. Consumers with thin hair fibers, moreover, want a perceptible strengthening of the hair fibers in association with hairstyle retention.

The object of the present invention, therefore, was to provide storage-stable powdered hair treatment agents for temporary shaping, which can be dispensed and applied precisely and simply, do not cause hair to stick together, and give the hair volume, strength, a fuller and natural feel, and a natural shine. The durability of the styling result should not be detrimentally affected.

It was determined that powdered cosmetic compositions based on a polyol-emulsifier mixture achieve the aforesaid object.

Furthermore, other desirable features and characteristics of the present invention will become apparent from the subsequent detailed description of the invention and the appended claims, taken in conjunction with this background of the invention.

### BRIEF SUMMARY OF THE INVENTION

Powdered cosmetic compositions, including 10 to 30% by weight of a hydrophobized metal oxide powder, 40 to 89% by weight of a polar solvent, and 0.001 to 8.0% by weight of a polymer fiber material.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present invention constitutes powdered cosmetic compositions, including
a) 10 to 30% by weight of a hydrophobized metal oxide powder,
b) 40 to 89% by weight of a polar solvent,
c) 0.001 to 8.0% by weight of a polymer fiber material.

The powdered compositions of the invention are present preferably in the form of core-shell particles, whose shell includes particles of at least one hydrophobized metal oxide powder and whose liquid core includes an organic polyol and optionally water.

Particles in the context of the invention are particles of solids present as grains (cf. DIN 66160: 1992-09).

Compositions whose particles are freely pourable under their own weight are powdered in the context of the invention (cf. DIN EN ISO 6186: 1998-08).

The powdered compositions of the invention are characterized in that the liquid core is released from the core-shell particles by mechanical stress on the core-shell particles, in particular by friction and/or pressure, and a liquid forms thereby from the powdered composition. Therefore, this is a powdered powder-to-liquid composition. The powdered compositions of the invention can be dispensed very simply. They can be distributed very uniformly in the hair, moreover, because the liquid core is released only under mechanical stress at the site of action and a selective wetting of the hair fibers is enabled. The powder can therefore first be carefully distributed in the hair and only then be stressed more greatly mechanically, for example, by selective massaging of the powder into the hair. As a result, the styling effect is produced only directly on the desired section of hair.

The employed powdered compositions include hydrophobized metal oxide. Preferred compositions are characterized in that they include the hydrophobized metal oxide powder, based on their total weight, in amounts of 10 to 25, preferably of 12 to 22% by weight. The optimum amount in this case depends primarily of the hydrophobicity of the employed silicon dioxide powder. The more hydrophobic the silicon dioxide powder, the less thereof is needed in order to obtain a stable powdered product.

The nature of the hydrophobized metal oxide is in principle not limited, provided it is assured that a powdered product forms upon intensive mixing with the liquid aqueous phase. Metal oxides that have been modified, at least on the surface of the particles, such that the modified particle is wetted less by water than the unmodified particle, are to be understood as hydrophobized in the context of the invention. Silanized, hydrophobized metal oxides are particularly preferred. At least one representative of the group formed by silanes, halosilanes, alkoxysilanes, and silazanes is preferably suitable according to the invention as a reagent for silanizing the metal oxide. Preferably suitable hydrophobized metal oxides of the hydrophobized metal oxide powder are selected according to the invention from at least one representative of the group formed by hydrophobized silicates, hydrophobized aluminum silicates, hydrophobized titanium dioxide, and hydrophobized silicon dioxide. Hydrophobized silicates have proven to be particularly suitable for preparing the cosmetic compositions of the invention, whereby pyrogenic silicic acid aftertreated by silanization or by reaction with polydimethylsiloxane has particular advantages.

The employed powdered compositions preferably include hydrophobized silicon dioxide. The nature of the hydrophobized silicon dioxide is in principle not limited, provided it is assured that a powdered product forms upon intensive mixing with polyol b), copolymer c), and optionally further ingredients.

Particularly preferably, the powdered composition of the invention includes as the hydrophobized metal oxide powder at least silanized, hydrophobized silicon dioxide.

Preferably, at least one representative of the group formed by silanes, halosilanes, alkoxysilanes, and silazanes is suitable according to the invention as a reagent for silanizing the silicon dioxide.

Preferred representatives of the group of silanes are hexa(C₁-C₂₀) alkyl disilanes, particularly hexamethyldisilane.

If a halosilane is used as the silylating agent, selected as the preferred halosilane is at least one compound from the group formed by the compounds
[(C₁-C₂₀)alkyl]_{z′}SiX_{(4-z′)}
X₃Si[(CH₂)ₙ—R]
X₂[(C₁-C₂₀)alkyl]Si(CH₂)ₙ—R
[(C₁-C₂₀)alkyl]_{(y′+1)}[R—(CH₂)ₙ]_{(2-y′)}SiX
   where
   X denotes a chlorine, bromine, or iodine atom,
   z′ is a number 1, 2, or 3,
   y′ is a number 0, 1, or 2,
   n is an integer from 1 to 20, and
   R stands for a group from
   (C₁-C₁₀) alkyl, aryl, (C₁-C₆) perfluoroalkyl, —NH₂, —N₃, —SCN, —CH═CH₂,

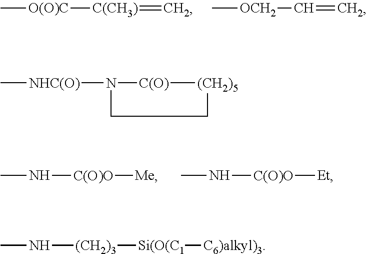

If an alkoxysilane is used as the silylating agent, selected as the preferred alkoxysilane is at least one compound from the group formed by the compounds
[(C₁-C₂₀)alkylO]_{z}Si(C₁-C₂₀)alkyl_{(4-z)}
[(C₁-C₂₀)alkylO]_{z}Si[(CH₂)ₙ—R]_{(4-z)}
[(C₁-C₂₀)alkylO]₂[(C₁-C₂₀)alkyl]Si(CH₂)ₙ—R
[(C₁-C₂₀)alkylO][(C₁-C₂₀)alkyl]₂Si(CH₂)ₙ—R
[(C₁-C₂₀)alkylO][(C₁-C₂₀)alkyl]Si(CH₂)ₙ—R₂
(C₁-C₂₀ alkyl)₃SiO—C(CH₃)═N—Si(C₁-C₂₀)alkyl₃
   where
   n is an integer from 1 to 20, and
   z denotes a number 1, 2, or 3,
   R stands for a group from
   (C₁-C₂₀) alkyl, aryl, (C₁-C₆) perfluoroalkyl, —NH₂, —N₃, —SCN, —CH═CH₂,

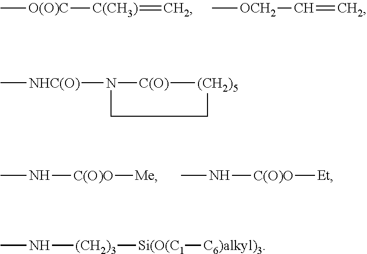

Selected as the preferred silazane is at least one compound from the class of disilazanes, in particular at least one compound from disilazanes of the formula

R′₂R″Si—NH—SiR′₂R″,

where
R′ denotes a (C₁-C₂₀) alkyl group and
R″ denotes a (C₁-C₂₀) alkyl group or a vinyl group. A particularly preferred silazane is hexamethyldisilazane.

All of the aforesaid alkyl groups, whether (C₁-C₆) alkyl, (C₁-C₁₀) alkyl, or (C₁-C₂₀) alkyl, can be both cyclic and linear or branched. Examples of alkyl groups usable according to the invention are methyl, ethyl, n-propyl, isopropyl, n-butyl, cyclopentyl, cyclohexyl, n-decyl, lauryl, myristyl, cetyl, stearyl, isostearyl, and behenyl.

An example of an aryl group of the invention is the phenyl group.

Examples of a (C₁-C₆) perfluoroalkyl group of the invention are trifluoromethyl, perfluoroethyl, perfluoropropyl, and perfluorohexyl.

Used preferably are hydrophobized silicon dioxides obtained by silanization of pyrogenic silicon dioxide.

Silanized, hydrophobized silicon dioxide are selected particularly preferably from at least one compound of the group formed by trimethyl silylate-coated silicon dioxide, dimethyl silylate-coated silicon dioxide, and octyl silylate-coated silicon dioxide.

Used preferably are hydrophobized silicon dioxides obtained by silanization of pyrogenic silicon dioxide.

A plurality of suitable hydrophobized silicon dioxides are commercially available. Recited as examples are Aerosil® R104 V, Aerosil® R106, Aerosil® R202, Aerosil® R805, Aerosil® R812, Aerosil® R812S, Aerosil® R972, and Aerosil® R8200, all from Degussa, and HDK® H2000, HDK® H2050, and HDK® H3004, all from Wacker.

It is particularly preferred to use the hydrophobized silicon dioxides that are obtainable under the names Aerosil® R202, Aerosil® R812S, or Aerosil® R972. It is very particularly preferred to use the silicon dioxide with the INCI name Silica Silylate, which is marketed by the company Degussa under the name Aerosil® R812S.

Preferred compositions of the invention therefore are characterized in that they include as the hydrophobized metal oxide powder a hydrophobized silicate, preferably a pyrogenic silicic acid aftertreated with polydimethylsiloxane. Suitable metal oxides with the INCI name “Silica Dimethicone Silylate” are marketed, for example, by the company Evonik under the trade name Aerosil® R202.

The particle diameter of the primary particles of preferred hydrophobized metal oxides is preferably less than 5 μm, particularly preferably less than 1 μm, and in particular between 1 and 50 nm.

Preferred furthermore are hydrophobized silicon dioxides that have a specific surface according to BET between 10 and 400 m²/g, preferably between 40 to 300 m²/g, and in particular 80 to 150 m²/g.

The powdered compositions of the invention include a polar solvent b) as a second essential component. Preferred compositions include the polar solvent, based on their total weight, in amounts of 42 to 88% by weight, preferably of 45 to 87% by weight.

Individual substances or substance mixtures can be used as the polar solvent. Preferred polar solvents, apart from water, are specific polyols. Preferred agents of the invention are notable in that they include less than four, preferably one to three, but in particular only one polyol. Suitable in this case for preparing cosmetic compositions are in particular polyols from the group comprising glycerol, 1,2-ethanediol, polyethylene glycol with MW>400, propanediol, butanediol, particularly 1,3-butanediol, hexanediol, particularly 1,6-hexanediol, sorbitol, threitol, erythritol, arabitol, altritol, ribitol, xylitol, galactitol, mannitol, iditol, and panthenol. Particularly suitable organic polyols are glycerol, sorbitol, and panthenol. These polyols can be converted into a powder form by relatively small amounts of hydrophobized metal oxide powder, usually with less than 10% by weight of metal oxide powder (based on the total weight of the powdered cosmetic composition). Preferred compositions of the invention are therefore characterized in that they include as the organic polyol at least one compound from the group comprising glycerol, sorbitol, and panthenol, preferably glycerol.

In summary, compositions are preferred that include as polar solvent b) at least one solvent from the group comprising water and glycerol, preferably water.

The weight proportion of the polar solvent in general and of the polyol specifically can vary over broad ranges.

Particularly preferred are compositions of the invention that primarily or exclusively include water as the polar solvent. Suitable preferred compositions are characterized in that, based on their total weight, they include
60 to 88% by weight, preferably 65 to 87% by weight of water, and
less than 10% by weight, preferably less than 5% by weight, preferably less than 1% by weight of glycerol.

In the compositions of the invention with a low polyol content, preferably a further polar solvent is added to the compositions by way of compensation, whereby the addition of water is particularly preferred. Preferred compositions are therefore characterized in that, based on their total weight, they include
12 to 30% by weight, preferably 15 to 25% by weight of organic polyol b) and
40 to 75% by weight, preferably 50 to 70% by weight of water.

When a high weight proportion of the polyol is used, the addition of further polar solvents, in particular the addition of water, is omitted as greatly as possible. Suitable compositions preferred according to the invention are characterized in that, based on their total weight, they include
60 to 88% by weight, preferably 65 to 87% by weight of organic polyol b) and
less than 10% by weight, primarily less than 5% by weight, preferably less than 1% by weight of water.

The addition of the polyol improves the mechanical properties of the compositions of the invention during storage and application and has advantageous cosmetic effects.

The composition of the invention includes 0.001 to 8.0% by weight of a polymer fiber material as a third essential component. A fiber refers to a structure which is flexible and thin in relation to its length and in which the ratio of the length to the diameter is preferably at least 3:1, primarily at least 10:1, particularly preferably at least 100:1, and in particular at least 1000:1. Fibers cannot absorb any pressure forces in the longitudinal direction but only tensile forces, because they bend under a pressure load. The weight proportion of polymer fiber material c) in terms of the total weight of the composition is preferably 0.01 to 5.0% by weight and in particular 0.1 to 1.0% by weight. The addition of the polymer fiber material increases the volume of a hairstyle set by means of the composition of the invention and strengthens the hair fibers. Preferred polymers of polymer fiber material c) are selected from the group of polyamide, viscose, and polypropylene.

Polyamides are linear polymers with regularly repeating amide bonds along the main chain. The amide group can be regarded as a condensation product of a carboxylic acid and an amine. Preferred polyamides are Nylon-6, prepared by ring opening polymerization of ε-caprolactam, and Nylon-66 prepared from hexamethylenediamine and adipic acid.

Chemical fibers that are spun industrially from cellulose with the aid of the viscose method are called viscose fibers. Cellulose is converted to cellulose xanthogenate in successive process steps by treatment with sodium hydroxide solution and reaction of the resulting alkali cellulose with carbon disulfide. The viscose spinning solution is produced by further addition of sodium hydroxide solution and is pumped through the holes of shower-like spinnerets into the spin bath. A viscose filament forms there per spinneret hole by coagulation. Viscose filament yarn forms by stretching and other processing steps and the combining of the individual filaments or viscose staple fibers by additional cutting.

Polypropylene is a partially crystalline thermoplastic from the group of polyolefins that can be processed into fibers, which are used, for example, in the textile industry.

The thickness of the individual fibers of the polymer fiber material is preferably 1 to 100 μm, preferably 10 to 80 μm, and in particular 15 to 60 μm.

The length of the individual fibers of the polymer fiber material is preferably 0.5 to 10 mm, preferably 1.0 to 8.0 mm, and in particular 1.5 to 5.0 mm.

Preferred compositions are characterized in that they include as an optional component a dye d) from the group of pigments, whereby the weight proportion of dye d) in terms of the total weight of the composition is preferably 0.1 to 8.0% by weight, primarily 0.2 to 6.0% by weight, and in particular 0.5 to 5.0% by weight.

In an embodiment preferred according to the invention, the inorganic or organic pigment is selected from the group formed by mineral pigments, metal pigments, pearlescent pigments, organic colored pigments, and pigment blacks. Provided the pigments are metal oxides, then these metal oxides are different from hydrophobized metal oxide powder a).

Inorganic pigments in this case comprise naturally occurring pigments, pigments obtainable by mechanical treatment, and synthetic pigments.

Mineral pigments or natural inorganic pigments include predominantly sulfides and oxides. Preferred natural pigments are ochre (Fe(OOH); Pigment Yellow 43), burnt sienna (Fe₂O₃; Pigment Red 102), umber (Fe₂O₃.xMnO2; Pigment Brown 7:x), cinnabar (β-HgS, PR 106), lapis lazuli (ultramarine, Na₆Al₆Si₆O₂₄.Na₂Sn; Pigment Blue 29), azurite (basic copper carbonate, Cu₃[OH/CO₃]₂; PB 30), green earth (FeO-containing silicate; Pigment Green 23), malachite (Cu₂[(OH)₂, CO₃]), and carbon black (carbon (graphite), Pigment Black 9).

The color-imparting synthetic iron oxides form a group of particularly preferred pigments. Particularly preferred representatives of this class of substances are Pigment Black 11 (CI No 77499), Pigment Brown 6 (CI No 77491), Pigment Red 101 (CI No 77491), Pigment Yellow 42 (CI No 77492), and mixtures of said pigments.

Synthetic inorganic pigments are obtained in particular by chemical and/or physical conversion (digestion, precipitation, annealing). These include in particular:
white pigments (titanium dioxide (TiO₂), Pigment White PW 6; zinc sulfide (ZnS), PW 7; zinc oxide (ZnO), PW 4; antimony white (Sb₂O₃), PW 11; lithopone (ZnS/BaSO₄), PW 5; white lead (2PbCO₃.Pb(OH)₂), PW 1),
secondarily white fillers (calcium carbonate, PW 18; talc, PW 26, and barium sulfate, PW 21);
black pigments (manganese black, spinel black, and pigment blacks (graphite-carbon));
luster pigments (absorption pigments, metal pigments, or metal effect pigments, and pearlescent pigments), and
inorganic colored pigments (iron oxide pigments, iron blue pigments, ultramarine pigments, and the lead chromate pigments less suitable on account of their toxicological properties, chromium oxide pigments, cadmium pigments, and bismuth vanadate pigments).

Pearlescent pigments are luster pigments that consist of several layers with a different refractive index. Pearlescent pigments preferred according to the invention are magnesium stearate, zinc stearate, and lithium stearate or ethylene glycol distearate or polyethylene terephthalate, as well as pearlescent pigments that consist substantially of mica, titanium dioxide (titanium dioxide-mica), bismuth chloride oxide, or guanine, and moreover can be coated with colored oxide layers (e.g., iron oxides or chromium oxides).

Pearlescent pigments with a mica base and mica/metal oxide base are pearlescent pigments particularly preferred according to the invention. Mica is a phyllosilicate. The most important representatives of these silicates are muscovite, phlogopite, paragonite, biotite, lepidolite, and margarite. To produce the pearlescent pigments in conjunction with metal oxide, mica, primarily muscovite or phlogopite, is coated with a metal oxide. Suitable metal oxides are, inter alia, TiO₂, Cr₂O₃, and Fe₂O₃. Interference pigments and colored luster pigments are obtained as pearlescent pigments preferred according to the invention by suitable coating. These pearlescent pigment types have additionally color effects apart from a glittering optical effect. Furthermore, the pearlescent pigments usable according to the invention include in addition a color pigment that does not derive from a metal oxide.

Very particularly preferred pearlescent pigments are pigments, marketed by the company Merck under the trade names Colorona®, whereby these include the pigments Colorona® red-brown (47-57% by weight of muscovite mica (KH₂(AlSiO₄)₃), 43-50% by weight of Fe₂O₃ (INCI: Iron Oxides CI 77491), <3% by weight of TiO₂ (INCI: Titanium Dioxide CI 77891), Colorona® Blackstar Blue (39-47% by weight of muscovite mica (KH₂(AlSiO₄)₃), 53-61% by weight of Fe₃O₄ (INCI: Iron Oxides CI 77499)), Colorona® Siena Fine (35-45% by weight of muscovite mica (KH₂(AlSiO₄)₃), 55-65% by weight of Fe₂O₃ (INCI: Iron oxides CI 77491)), Colorona® Aborigine Amber (50-62% by weight of muscovite mica (KH₂(AlSiO₄)₃), 36-44% by weight of Fe₃O₄ (INCI: Iron Oxides CI 77499), 2-6% by weight of TiO₂ (INCI: Titanium Dioxide CI 77891)), Colorona® Patagonian Purple (42-54% by weight of muscovite mica (KH₂(AlSiO₄)₃), 26-32% by weight of Fe₂O₃ (INCI: Iron Oxides CI 77491), 18-22% by weight of TiO₂ (INCI: Titanium Dioxide CI 77891), 2-4% by weight of Prussian blue (INCI: Ferric Ferrocyanide CI 77510)), Colorona® Chameleon (40-50% by weight of muscovite mica (KH₂(AlSiO₄)₃), 50-60% by weight of Fe₂O₃ (INCI: Iron Oxides CI 77491)), and Silk® Mica (>98% by weight of muscovite mica (KH₂(AlSiO₄)₃)).

Powdered metals or metal alloys are referred to as metal pigments or metal effect pigments. Particularly advantageous according to the invention are aluminum pigments (aluminum bronzes) and brass pigments (so-called gold bronzes), therefore copper/aluminum alloys or copper/zinc alloys, whereby the term “bronze” in this context is not to be regarded in the narrow chemical sense. Exemplary metal pigments are:
Aluminum bronzes (Al; color silver);
Gold bronzes (Cu, Cu—Al, or Cu—Zn alloy; natural shades)
   (Cu; copper color)
   (90% Cu-10% Zn; pale gold)
   (85% Cu-15% Zn; rich pale gold)
   (70% Cu-30% Zn; rich gold)
Silver bronzes (Cu—Zn—Ni; silver)
Oxidized bronzes (oxidized Cu—Zn; tempering colors such as citron, fire red, sea green)
Patent bronzes (Cu—Zn—(Ni)+dyes; various colors)

Organic pigments suitable according to the invention are in particular synthetic organic pigments. Natural organic pigments are present in the agent of the invention preferably in combination with further pigments. Natural organic pigments in this case include gamboge, indigo, and chlorophyll.

Synthetic organic pigments are particularly preferred according to the invention, however. These include in particular azo pigments and polycyclic pigments.

Preferred azo pigments are monoazo yellow pigments, BONA (β (beta)-ortho-naphtholcarboxylic acid) pigments, β-naphthol pigments, naphthol AS pigments, benzimidazolone pigments, pyrazoloquinazolone pigments, diaryl yellow pigments, bisacetacetic acid arylamide pigments, disazopyrazolone pigments, and disazo condensation pigments.

Suitable yellow pigments are monoazo yellow pigments, which are produced from substituted anilines as the diazo component and methylene-active compounds, such as acetacetic acid arylamides or substituted pyrazolones, as the coupling component. The color palette extends from green-tinted to red-tinted yellow, and in the case of pyrazolones to orange tones (monoazo orange pigments or monoazo pyrazolone pigments). These include the preferred PY 1, PY 74, and PY 97.

Preferred β-naphthol pigments are PO 2, PO 5, PO 17, PO 17:1, PR 1 (para red), PR 3 (toluidine red), PR 4, PR 6, PR 50:1, PR 51, PR 53:1, PR 53, PR 68, PR 49:1, and PR 49:2.

Preferred BONA (β (beta)-ortho-naphtholcarboxylic acid) pigments are PR 48:1, PR 48:2, PR 48:3, PR 48:4, PR 48:5, PR 52:1, PR 52:2, PR 57, PR 57:1, PR 58:2, PR 58:4, PR 64, and PR 64:1. Particularly preferred in this case are PR 48:1 and PR 52:1 and in particular PR 57 or PR 57:1 (PR 57:1 stands for the Ca²⁺ salt of PR 57).

Preferred naphthol AS pigments are PO 38, PO 74, PR 2, PR 5, PR 14, PR 112, PR 146, PR 147, PR 170, PR 184, PR 187, PR 188, PR 210, and PR 253.

Preferred benzimidazolone pigments are PY 151, PY 175, PO 36, PO 60, PR 175, PR 176, PR 185, PR 208, PV 32, and PBr 25.

Preferred pyrazoloquinazolone pigments are PO 67 and PR 251.

Among the azo pigments, diaryl yellow pigments in particular are suitable as yellow pigments according to the invention. Preferred diaryl yellow pigments are PY (Pigment Yellow) 12, PY 13, PY 14, PY 17, PY 55, PY 63, PY 63, PY 81, PY 83, PY 87, PY 90, PY 106, PY 113, PY 114, PY 121, PY 124, PY 126, PY 127, PY 136, PY 152, PY 170, PY 171, PY 172, PY 174, PY 176, PY 188, and PO (Pigment Orange) 15, PO 16, and PO 44. PY 12 and PY 13 are particularly preferred.

Preferred disazopyrazolone pigments are PO 13, PO 34, PR 37, PR 38, and PR 111.

Preferred disazo condensation pigments are PY 93, PY 94, PY 95, PY 128, PO 31, PR 144, PR 166, PR 221, PR 242, PR 262, PBr 23, PBr 41, and PBr 42.

Polycyclic pigments in the context of the invention are all organic non-azo pigments that have at least one aromatic or heteroaromatic ring system. For a more precise characterization, it is typical to subdivide further the compound class of the polycyclic pigments. The skilled artisan in this case is familiar with the following:
a) Strictly polycyclic pigments such as, for example, phthalocyanine pigments, quinacridone pigments, triarylmethane pigments, triphenylmethane pigments, dye salts with complex anions, and dioxazine pigments;
b) Non-azo pigments such as, for example, metal complex pigments, azomethine pigments, bis(azomethine) pigments, isoindolinone pigments, isoindoline pigments, quinophthalone pigments, diketopyrrolopyrrole pigments, and aniline black;
c) Vat dye-derived pigments such as, for example, perylene pigments, naphthalene tetracarboxylic acid pigments, thioindigo pigments, anthraquinone pigments, aminoanthraquinone pigments, anthrapyrimidine pigments, indanthrone pigments, flavanthrone pigments, pyranthrone pigments, and anthanthrone pigments.

Polycyclic pigments suitable according to the invention are the so-called phthalocyanine pigments. This refers to porphyrin metal complexes. Particularly advantageous according to the invention are the copper phthalocyanine pigments, which exist in different colors and modifications. A distinction is made by color between phthalo blue and phthalo green types, whereby the green color is generally produced by additional halogenation of the porphyrin ring. In addition, cobalt or aluminum-containing complexes can also be used. Phthalocyanines preferred according to the invention, however, are the copper phthalocyanine pigments, in particular the phthalo blue pigments PB 15 (C₃₂H16CuN₈), PB:1, PB 15:2, PB 15:3, and PB 15:4, whereby the numbers which follow in each case signify a different modification and/or a different halogenation component in the porphyrin, and the phthalo green pigments PG 17 (C₃₂HC₁₅CuN₈) and PG 36 (C₃₂Br₆C₁₀CuN₈).

Further pigments advantageous according to the invention are the so-called pigment blacks (carbon black, also carbon pigments). These are understood to be finely divided industrial carbon blacks which are used as black pigments and for UV stabilization. Pigment blacks are exceedingly resistant to chemicals and are notable for high light and weathering resistance. Because of the very high color depth and strength and other specific properties, pigment blacks are the black pigments preferred according to the invention (Pigment Black PBI 7).

In a further embodiment, the compositions of the invention are characterized in that the inorganic or organic pigment is selected from the group formed by Pigment Yellow 12 and 13 (PY 12/PY 13), Pigment Red 57 (PR 57), Pigment Blue 15 (PB 15), Pigment Black 7 (PBI 7), Pigment White 6 (PW 6), and aluminum and brass pigments.

Preferred compositions are characterized in that dye d) is selected from the group of water-insoluble pigments, whereby the weight proportion of dye d) in terms of the total weight of the composition is 0.1 to 8.0% by weight, preferably 0.2 to 6.0% by weight, and in particular 0.5 to 5.0% by weight.

Particularly preferred compositions of the invention are characterized in that polymer fiber material c) and dye d) are present at least partially in the form of a composite material. Particularly preferred are in particular compositions in which
at least 20% by weight, primarily at least 50% by weight, preferably at least 80% by weight, particularly preferably at least 90% by weight, and in particular the entire polymer fiber material present and
at least 2% by weight, primarily 2 to 80% by weight, preferably 5 to 60% by weight, and in particular 10 to 40% by weight of dye d)
   are present in the form of composite materials of polymer fiber material c) and dye d).

Preferred combinations of polymer fiber material c) with dyes d), which for their part are preferably present in the form of composites, are
Nylon-6 and iron oxides CI 77491, Nylon-6 and iron oxides CI 77492, Nylon-6 and iron oxides CI 77499, Nylon-6 and titanium dioxide CI 77891, Nylon-6 and iron oxides CI 77499, Nylon-6 and ferric ferrocyanide CI 77510, Nylon-6 and Pigment Yellow 12, Nylon-6 and Pigment Yellow 13, Nylon-6 and Pigment Red 57, Nylon-6 and Pigment Blue 15, Nylon-6 and Pigment Black 7, Nylon-6 and Pigment White 6, as well as
Nylon-66 and iron oxides CI 77491, Nylon-66 and iron oxides CI 77492, Nylon-66 and iron oxides CI 77499, Nylon-66 and titanium dioxide CI 77891, Nylon-66 and iron oxides CI 77499, Nylon-66 and ferric ferrocyanide CI 77510, Nylon-66 and Pigment Yellow 12, Nylon-66 and Pigment Yellow 13, Nylon-66 and Pigment Red 57, Nylon-66 and Pigment Blue 15, Nylon-66 and Pigment Black 7, Nylon-66 and Pigment White 6

The aforesaid combinations can include silicate as an optional component.

Preferred additives of the compositions of the invention, which include apart from a polymer fiber material c) further also a fiber material d), are obtainable, for example, from the company Kobo Products under the trade names:
Nylon Cut Fiber 6D (INCI: Nylon-66, Titanium Dioxide, Silica)
Nylon-66 Fiber 20D (INCI: Nylon-66, Titanium Dioxide)
Nylon-66 Fiber 3D (INCI: Nylon-66, Titanium Dioxide)
PP Fiber 6D05 (INCI: Polypropylene, Silica)
PP Fiber 6D2 (INCI: Polypropylene, Silica)
NFCB-10D-2T and NFCB-10D-2R (INCI: Nylon-6, Silica, Black 2)
NFCB-10D-4R (INCI: Nylon-6, Black 2)
NFBL-10D-2R and NFBL-10D-4R (INCI Nylon-6, Iron Oxides (CI: 77499), Triethoxycaprylylsilane, Silica);
NFBR-10D-2R and NFBR-10D-2T (INCI: Nylong-6, Iron Oxides (CI 77499), Iron Oxides (CI 77491), Iron Oxides (CI 77492), Silica);
RFCB-10D-2R (INCI: Rayon, Silica, Black 2), and obtainable from the company Daito Kasei under the trade names:
NFW (INCI: Nylon-6, Silica, Titanium Dioxide);
NFCB (INCI: Nylon-6, Silica, Pigment Black 7 (CI 77266)).

An optional component of the compositions of the invention are copolymers e), which are obtained by polymerizing the monomers N-tert-octylacrylamide, acrylic acid, and tert-butylaminoethyl methacrylic acid. Preferred copolymers e) consist of at least 90% by weight, preferably at least 95% by weight, and particularly at least 97% by weight of the monomers, N-tert-octyl acrylamide, acrylic acid, and tert-butylaminoethyl methacrylic acid. Particularly preferred copolymers e) were obtained exclusively from the monomers, N-tert-octyl acrylamide, acrylic acid, and tert-butylaminoethyl methacrylic acid.

The previously described copolymers e) are, for example, marketed under the name Amphomer® (INCI name: Octylacrylamide/Acrylates/Butylaminoethyl Methacrylate Copolymer) by the company National Starch.

Preferred compositions of the invention are characterized in that the weight proportion of copolymer e) in terms of the total weight of the composition is 0.01 to 15% by weight, preferably 0.02 to 12% by weight, primarily 0.03 to 8.0% by weight, and in particular of 0.05 to 1.0% by weight.

In summary, such compositions are preferred that, based on their total weight, include 0.01 to 15% by weight, preferably 0.02 to 12% by weight, primarily 0.03 to 8.0% by weight, and in particular of 0.05 to 1.0% by weight of at least one copolymer e) from the monomers:
b1) N-tert-octylacrylamide,
b2) acrylic acid,
b3) tert-butylaminoethyl methacrylic acid,
b4) and optionally further monomers.

The composition of some preferred cosmetic agents can be obtained from the following tables (data are given in % by weight, based on the total weight of the cosmetic agent, unless otherwise stated).

| | Formula 1 | Formula 2 | Formula 3 | Formula 4 | Formula 5 |
|---|---|---|---|---|---|
| Hydrophobized metal | 10 to 30 | 10 to 25 | 10 to 25 | 12 to 22 | 12 to 22 |
| oxide powder | | | | | |
| Polar solvent | 40 to 89 | 42 to 88 | 42 to 88 | 45 to 87 | 45 to 87 |
| Polymer fiber material | 0.001 to 8.0 | 0.01 to 5.0 | 0.01 to 5.0 | 0.1 to 1.0 | 0.1 to 1.0 |
| Optional additives | To 100 | To 100 | To 100 | To 100 | To 100 |

| | Formula 6 | Formula 7 | Formula 8 | Formula 9 | Formula 10 |
|---|---|---|---|---|---|
| Silica silylates | 10 to 30 | 10 to 25 | 10 to 25 | 12 to 22 | 12 to 22 |
| Polar solvent | 40 to 89 | 42 to 88 | 42 to 88 | 45 to 87 | 45 to 87 |
| Polymer fiber material | 0.001 to 8.0 | 0.01 to 5.0 | 0.01 to 5.0 | 0.1 to 1.0 | 0.1 to 1.0 |
| Optional additives | To 100 | To 100 | To 100 | To 100 | To 100 |

| | Formula 11 | Formula 12 | Formula 13 | Formula 14 | Formula 15 |
|---|---|---|---|---|---|
| Silica silylates | 10 to 30 | 10 to 25 | 10 to 25 | 12 to 22 | 12 to 22 |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polymer fiber material | 0.001 to 8.0 | 0.01 to 5.0 | 0.01 to 5.0 | 0.1 to 1.0 | 0.1 to 1.0 |
| Optional additives | To 100 | To 100 | To 100 | To 100 | To 100 |

| | Formula 16 | Formula 17 | Formula 18 | Formula 19 | Formula 20 |
|---|---|---|---|---|---|
| Silica dimethicone | 10 to 30 | 10 to 25 | 10 to 25 | 12 to 22 | 12 to 22 |
| silylates | | | | | |
| Polar solvent | 40 to 89 | 42 to 88 | 42 to 88 | 45 to 87 | 45 to 87 |
| Polymer fiber material | 0.001 to 8.0 | 0.01 to 5.0 | 0.01 to 5.0 | 0.1 to 1.0 | 0.1 to 1.0 |
| Optional additives | To 100 | To 100 | To 100 | To 100 | To 100 |

| | Formula 21 | Formula 22 | Formula 23 | Formula 24 | Formula 25 |
|---|---|---|---|---|---|
| Silica dimethicone | 10 to 30 | 10 to 25 | 10 to 25 | 12 to 22 | 12 to 22 |
| silylates | | | | | |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polymer fiber material | 0.001 to 8.0 | 0.01 to 5.0 | 0.01 to 5.0 | 0.1 to 1.0 | 0.1 to 1.0 |
| Optional additives | To 100 | To 100 | To 100 | To 100 | To 100 |

| | Formula 26 | Formula 27 | Formula 28 | Formula 29 | Formula 30 |
|---|---|---|---|---|---|
| Hydrophobized metal | 10 to 30 | 10 to 25 | 10 to 25 | 12 to 22 | 12 to 22 |
| oxide powder | | | | | |
| Polar solvent | 40 to 89 | 42 to 88 | 42 to 88 | 45 to 87 | 45 to 87 |
| Polymer fiber material | 0.001 to 8.0 | 0.01 to 5.0 | 0.01 to 5.0 | 0.1 to 1.0 | 0.1 to 1.0 |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 31 | Formula 32 | Formula 33 | Formula 34 | Formula 35 |
|---|---|---|---|---|---|
| Silica silylates | 10 to 30 | 10 to 25 | 10 to 25 | 12 to 22 | 12 to 22 |
| Polar solvent | 40 to 89 | 42 to 88 | 42 to 88 | 45 to 87 | 45 to 87 |
| Polymer fiber material | 0.001 to 8.0 | 0.01 to 5.0 | 0.01 to 5.0 | 0.1 to 1.0 | 0.1 to 1.0 |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 36 | Formula 37 | Formula 38 | Formula 39 | Formula 40 |
|---|---|---|---|---|---|
| Silica silylates | 10 to 30 | 10 to 25 | 10 to 25 | 12 to 22 | 12 to 22 |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polymer fiber material | 0.001 to 8.0 | 0.01 to 5.0 | 0.01 to 5.0 | 0.1 to 1.0 | 0.1 to 1.0 |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 41 | Formula 42 | Formula 43 | Formula 44 | Formula 45 |
|---|---|---|---|---|---|
| Silica dimethicone | 10 to 30 | 10 to 25 | 10 to 25 | 12 to 22 | 12 to 22 |
| silylates | | | | | |
| Polar solvent | 40 to 89 | 42 to 88 | 42 to 88 | 45 to 87 | 45 to 87 |
| Polymer fiber material | 0.001 to 8.0 | 0.01 to 5.0 | 0.01 to 5.0 | 0.1 to 1.0 | 0.1 to 1.0 |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 46 | Formula 47 | Formula 48 | Formula 49 | Formula 50 |
|---|---|---|---|---|---|
| Silica dimethicone | 10 to 30 | 10 to 25 | 10 to 25 | 12 to 22 | 12 to 22 |
| silylates | | | | | |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polymer fiber material | 0.001 to 8.0 | 0.01 to 5.0 | 0.01 to 5.0 | 0.1 to 1.0 | 0.1 to 1.0 |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 51 | Formula 52 | Formula 53 | Formula 54 | Formula 55 |
|---|---|---|---|---|---|
| Hydrophobized metal | 10 to 30 | 10 to 25 | 10 to 25 | 12 to 22 | 12 to 22 |
| oxide powder | | | | | |
| Polar solvent | 40 to 89 | 42 to 88 | 42 to 88 | 45 to 87 | 45 to 87 |
| Polyamide fiber | 0.001 to 8.0 | 0.01 to 5.0 | 0.01 to 5.0 | 0.1 to 1.0 | 0.1 to 1.0 |
| material | | | | | |
| Optional additives | To 100 | To 100 | To 100 | To 100 | To 100 |

| | Formula 56 | Formula 57 | Formula 58 | Formula 59 | Formula 60 |
|---|---|---|---|---|---|
| Silica silylates | 10 to 30 | 10 to 25 | 10 to 25 | 12 to 22 | 12 to 22 |
| Polar solvent | 40 to 89 | 42 to 88 | 42 to 88 | 45 to 87 | 45 to 87 |
| Polyamide fiber | 0.001 to 8.0 | 0.01 to 5.0 | 0.01 to 5.0 | 0.1 to 1.0 | 0.1 to 1.0 |
| material | | | | | |
| Optional additives | To 100 | To 100 | To 100 | To 100 | To 100 |

| | Formula 61 | Formula 62 | Formula 63 | Formula 64 | Formula 65 |
|---|---|---|---|---|---|
| Silica silylates | 10 to 30 | 10 to 25 | 10 to 25 | 12 to 22 | 12 to 22 |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polyamide fiber | 0.001 to 8.0 | 0.01 to 5.0 | 0.01 to 5.0 | 0.1 to 1.0 | 0.1 to 1.0 |
| material | | | | | |
| Optional additives | To 100 | To 100 | To 100 | To 100 | To 100 |

| | Formula 66 | Formula 67 | Formula 68 | Formula 69 | Formula 70 |
|---|---|---|---|---|---|
| Silica dimethicone | 10 to 30 | 10 to 25 | 10 to 25 | 12 to 22 | 12 to 22 |
| silylates | | | | | |
| Polar solvent | 40 to 89 | 42 to 88 | 42 to 88 | 45 to 87 | 45 to 87 |
| Polyamide fiber | 0.001 to 8.0 | 0.01 to 5.0 | 0.01 to 5.0 | 0.1 to 1.0 | 0.1 to 1.0 |
| material | | | | | |
| Optional additives | To 100 | To 100 | To 100 | To 100 | To 100 |

| | Formula 71 | Formula 72 | Formula 73 | Formula 74 | Formula 75 |
|---|---|---|---|---|---|
| Silica dimethicone | 10 to 30 | 10 to 25 | 10 to 25 | 12 to 22 | 12 to 22 |
| silylates | | | | | |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polyamide fiber | 0.001 to 8.0 | 0.01 to 5.0 | 0.01 to 5.0 | 0.1 to 1.0 | 0.1 to 1.0 |
| material | | | | | |
| Optional additives | To 100 | To 100 | To 100 | To 100 | To 100 |

| | Formula 76 | Formula 77 | Formula 78 | Formula 79 | Formula 80 |
|---|---|---|---|---|---|
| Hydrophobized metal | 10 to 30 | 10 to 25 | 10 to 25 | 12 to 22 | 12 to 22 |
| oxide powder | | | | | |
| Polar solvent | 40 to 89 | 42 to 88 | 42 to 88 | 45 to 87 | 45 to 87 |
| Polyamide fiber | 0.001 to 8.0 | 0.01 to 5.0 | 0.01 to 5.0 | 0.1 to 1.0 | 0.1 to 1.0 |
| material | | | | | |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 81 | Formula 82 | Formula 83 | Formula 84 | Formula 85 |
|---|---|---|---|---|---|
| Silica silylates | 10 to 30 | 10 to 25 | 10 to 25 | 12 to 22 | 12 to 22 |
| Polar solvent | 40 to 89 | 42 to 88 | 42 to 88 | 45 to 87 | 45 to 87 |
| Polyamide fiber | 0.001 to 8.0 | 0.01 to 5.0 | 0.01 to 5.0 | 0.1 to 1.0 | 0.1 to 1.0 |
| material | | | | | |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 86 | Formula 87 | Formula 88 | Formula 89 | Formula 90 |
|---|---|---|---|---|---|
| Silica silylates | 10 to 30 | 10 to 25 | 10 to 25 | 12 to 22 | 12 to 22 |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polyamide fiber | 0.001 to 8.0 | 0.01 to 5.0 | 0.01 to 5.0 | 0.1 to 1.0 | 0.1 to 1.0 |
| material | | | | | |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 91 | Formula 92 | Formula 93 | Formula 94 | Formula 95 |
|---|---|---|---|---|---|
| Silica dimethicone | 10 to 30 | 10 to 25 | 10 to 25 | 12 to 22 | 12 to 22 |
| silylates | | | | | |
| Polar solvent | 40 to 89 | 42 to 88 | 42 to 88 | 45 to 87 | 45 to 87 |
| Polyamide fiber | 0.001 to 8.0 | 0.01 to 5.0 | 0.01 to 5.0 | 0.1 to 1.0 | 0.1 to 1.0 |
| material | | | | | |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

| | Formula 96 | Formula 97 | Formula 98 | Formula 99 | Formula 100 |
|---|---|---|---|---|---|
| Silica dimethicone | 10 to 30 | 10 to 25 | 10 to 25 | 12 to 22 | 12 to 22 |
| silylates | | | | | |
| Water | 40 to 75 | 40 to 75 | 50 to 70 | 50 to 70 | 50 to 70 |
| Glycerol | 12 to 30 | 12 to 30 | 15 to 25 | 15 to 25 | 15 to 25 |
| Polyamide fiber | 0.001 to 8.0 | 0.01 to 5.0 | 0.01 to 5.0 | 0.1 to 1.0 | 0.1 to 1.0 |
| material | | | | | |
| Pigment | 0.1 to 8.0 | 0.1 to 8.0 | 0.2 to 6.0 | 0.2 to 6.0 | 0.5 to 5.0 |
| Optional additives | To 100 | To 100 | To 100 | To 100 | To 100 |

The cosmetic compositions of the invention can include further auxiliary and care substances and additives. The weight proportion of the further ingredients, included in the powdered compositions of the invention, apart from components a) to c), in particular the further auxiliary and care substances and additives included in these compositions, in terms of the total weight of the powdered compositions of the invention is preferably less than 10% by weight, preferably less than 5.0% by weight, particularly preferably less than 2.0% by weight, and in particular less than 1.0% by weight. The weight proportion of these auxiliary and care substances and additives in the total weight of the cosmetic agents of the invention can be, for example, 0.001 to 2% by weight, in particular 0.01 to 0.5% by weight.

The powdered compositions of the invention can be packaged in any containers, provided it is assured that the mechanical stress on the powder during the removal of the composition does not lead to liquefaction. Suitable are, for example, jars, bottles, or Tetra Paks as well, whereby the container can be designed, for example, with a pouring and dispensing means.

A further subject of the present invention is the use of a cosmetic composition of the invention for the temporary reshaping of keratin-containing fibers, in particular human hair. When the powdered composition is used for the temporary reshaping of keratinic fibers, preferably first the desired quantity of powdered composition is removed from the container. The composition can be applied directly onto the keratinic fibers to be treated or else, for example, onto one's hand. In the first case, the applied powder can be exposed to a mechanical stress directly on the keratinic fibers, for example, by means of one's hands, with the result that the liquid aqueous phase is released directly onto the fibers. If the powdered composition is first placed onto one's hand, it can then first be carefully distributed in the hair and only then in turn be subjected to greater mechanical stress, for example, by controlled massaging of the powder into the hair. The liquid aqueous phase is thereby released on the hair.

It is, of course, also possible to rub the powdered composition on one's hand and only then to apply the resulting liquid or pasty agent to the keratinic fibers. This procedure is not preferred, however, because an essential advantage of the powdered consistency of the styling agent, namely, good distribution capability, is thereby sacrificed. The powdered composition can, of course, also be applied using an aid, for example, a brush, a sponge, cloth, a hairbrush, or a comb.

A further subject of the present invention is a method for the temporary reshaping of keratin-containing fibers, in particular human hair, in which method the keratinic fibers are acted upon by a cosmetic composition of the invention and their shape is temporarily retained, characterized in that before, during, or after application to the keratinic fibers, a plastically deformable mass is formed from the cosmetic composition by the action of a force.

### Examples

The powdered styling agents V1 to V6 were prepared as described below (quantities are given in % by weight):

| | V1 | V2 | V3 | V4 | V5 | V6 |
|---|---|---|---|---|---|---|
| Silica silylates | 17 | 17 | 17 | 17 | 17 | 17 |
| Water | To 100 | To 100 | To 100 | To 100 | To 100 | To 100 |
| Glycerol | 20 | 19 | 18 | 19.5 | 20 | 19 |
| Mica Black | 3 | 3 | 2.5 | 3 | 2 | 2 |
| Nylon-6, Iron | 0.5 | 0.5 | — | — | — | — |
| Oxide | | | | | | |
| Nylon-6, | — | — | 0.5 | 0.5 | 0.5 | 0.5 |
| Silica, | | | | | | |
| Iron Oxide | | | | | | |

A stable powder was formed by mixing of the components. The thus obtained finished styling powder was filled into polyethylene bottles.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A powdered cosmetic composition, comprising
a) 10 to 30% by weight of a hydrophobized metal oxide powder,
b) 40 to 89% by weight of a polar solvent,
c) 0.001 to 8.0%/by weight of a polymer fiber material.

2. The composition according to claim 1, wherein the hydrophobized metal oxide powder comprises 10 to 25 wt % of the total composition.

3. The composition according to claim 1, wherein the hydrophobized metal oxide powder comprises 12 to 22 wt % of the total composition.

4. The composition according to claim 1, wherein the hydrophobized metal oxide powder is a hydrophobized silicate obtained by silanization of pyrogenic silicon dioxide.

5. The composition according claim 1, wherein the polar solvent b) comprises 42 to 88 wt % of the composition.

6. The composition according to claim 1, wherein the polar b) comprises 45 to 87% by weight of polar solvent b).

7. The composition according to claim 1, wherein the polar solvent b) is water or glycerol.

8. The composition according to claim 1, wherein the polar solvent b) is water.

9. The composition according to claim 1, wherein the composition comprises, based on its total weight, 12 to 30% by weight organic polyol and 40 to 5 wt % water.

10. The composition according to claim 1, wherein the composition comprises, based on its total weight 15 to 25 wt % of the organic polyol and 50 to 70 wt % of water.

11. The composition according to claim 1, wherein polymer fiber material c) comprises 0.01 to 5.0 wt % of the composition.

12. The composition according to claim 1, wherein the polymer fiber material c) comprises 0.1 to 1.0 wt % of the composition.

13. The composition according to claim 1, wherein the polymer fiber material c) is selected from the group consisting of a polyamide, a viscose, and a polypropylene.

14. The composition according to claim 1, wherein the dye d) is a pigment and comprises 0.1 to 8.0% of the composition.

15. The composition according claim 1, wherein the composition comprises, based on its total weight, 0.01 to 15% by weight of at least one copolymer e) including the monomers
a. N-tert-octylacrylamide,
b. acrylic acid,
c. tert-butylaminoethyl methacrylic acid,
d. and optionally further monomers.

16. The composition according claim 1, wherein the composition comprises, based on its total weight, 0.03 to 8.0% by weight of at least one copolymer e) including the monomers
a. N-tert-octylacrylamide,
b. acrylic acid,
c. tert-butylaminoethyl methacrylic acid,
d. and optionally further monomers.

17. The composition according claim 1, wherein the composition comprises, based on its total weight, 0.05 to 1.0% by weight of at least one copolymer e) including the monomers
a. N-tert-octylacrylamide,
b. acrylic acid,
c. tert-butylaminoethyl methacrylic acid,
d. and optionally further monomers.

18. A method for the temporary reshaping of keratin-containing fibers, comprising applying to the keratin-containing fibers a composition according to claim 1, wherein before, during, or after application to the keratinic fibers, a plastically deformable mass is formed from the cosmetic composition by the action of a force during.
